(19) United States
(12) Patent Application Publication
(10) Pub. No.: US 2004/0236286 A1
(43) Pub. Date: Nov. 25, 2004

(54) One-to-many infiltration tubing

(76) Inventor: Jeffrey A. Klein, San Juan Capistrano, CA (US)

Correspondence Address: Kit M. Stetina, STETINA BRUNDA GARRED & BRUCKER, Suite 250, 75 Enterpise, Aliso Viejo, CA, 92656, US

(21) Appl. No.: 10/443,509

(22) Filed: May 22, 2003

(51) Int. Cl. A61M 25/00; A61M 5/00

(52) U.S. Cl. 604/258; 604/284

(57) ABSTRACT

An infiltration tubing having a one-to-many connector is disclosed herein. The one-to-many connector includes a Y-shaped body. The Y-shaped body includes a proximal inlet configured to connect to a fluid source (e.g., an IV bag or bottle) and at least two distal outlets. Each distal outlet is configured to connect to a respective fluid delivery device (e.g., an infiltration cannula, a catheter or a hypodermic needle) for infiltrating fluid into a patient.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND OF THE INVENTION

The present invention relates in general to infiltration tubing and in particular to a one-to-many infiltration tubing.

Infiltration tubing is used for infiltrating fluids into a patient, for example for providing local anesthesia in order to perform a surgical procedure, such as liposuction. Typically, a single source of fluid is transported through the tubing and out a single fluid delivery device, such as a needle or catheter, and into the patient. There are presently tubing connectors that incorporate a many-to-one branching design that allows two or more separate types of intravenous (IV) fluids (contained in two or more streams) to be carried by separate IV lines to be combined and funneled through a single IV line that is then infused into a single vein through an intravenous catheter. For example, anesthesia and nutrients could be delivered from separate sources to a patient through a single delivery device.

There are some situations in which it is desirable to simultaneously deliver fluid from a single source to multiple sites on a single patient, for example, the infiltration of tumescent local anesthesia. If the local anesthesia (from a single source) could be delivered simultaneously to multiple sites (e.g., through multiple delivery devices, such as needles, cannulas or catheters), the time required to complete the infiltration could be dramatically decreased. Such a dramatic decrease in the infiltration time would in turn greatly decrease the patient's discomfort.

Presently, if it is desired to infiltrate fluid from a single source into multiple sites on a single patient (as described above), the infiltration device (e.g., needle or cannula) must be removed and reinserted in the new location. This temporarily terminates the infiltration process. Thus, using prior art devices, multiple sources containing the same fluid (e.g., local anesthetic), and multiple peristaltic infiltration pumps or multiple peristaltic pump heads on a single infiltration pump would be required in order to infiltrate at multiple sites without terminating the infiltration process.

Therefore, there is a need for a system of performing an infiltration process using fluid from a single source and infiltrating the fluid into multiple patient sites without terminating the infiltration process while the needle or cannula is removed and inserted into the new patient site, and without requiring the use of multiple infiltration pumps.

### BRIEF SUMMARY OF THE INVENTION

An infiltration tubing having a one-to-many connector is disclosed herein. The one-to-many connector includes a Y-shaped body. The Y-shaped body includes a proximal inlet configured to connect to a fluid source (e.g., an IV bag or bottle) and at least two distal outlets. Each distal outlet is configured to connect to a respective fluid delivery device (e.g., an infiltration cannula, a catheter or a hypodermic needle) for infiltrating fluid into a patient.

An IV tube may be disposed between the proximal inlet and the fluid source.

The at least two distal outlets may be substantially parallel in relation to each other. Alternatively, the two distal outlets may be in an angular relationship of less than 180 degrees with respect to each other.

The proximal inlet may be attached to a proximal inlet female connector and the fluid source may include a fluid source male connector with the proximal inlet female connector configured to connect to the fluid source male connector. The proximal inlet female connector may be attached to the proximal inlet via a short tubing. The short tubing may be glued to the proximal inlet female connector and the proximal inlet.

The proximal inlet female connector and the fluid source male connector may be luer connectors. The proximal inlet female connector and the fluid source male connector may have a luer slip connection or they may have a luer lock connection

Each of the at least two distal outlets may be configured to connect to the respective fluid delivery device with a distal tube disposed therebetween. Each of the at least two distal outlets may be glued to the respective distal tube.

Each of the at least two distal outlets may include a distal outlet male connector. Each of the respective distal tubes may include a distal tube female connector with each of the distal outlet male connectors configured to connect to a respective distal tube female connector. The distal outlet male connectors and the distal tube female connectors may be luer connectors which have a slip luer connection or a luer lock connection.

The distal tubing may include an on-off switch configured to temporarily stop all flow through the distal tubing. The on-off switch may be a clamp or a stopcock.

### BRIEF DESCRIPTION OF THE DRAWINGS

These as well as other features of the present invention will become more apparent upon reference to the drawings wherein:

FIG. 1 is a side elevation view of a one-to-many infiltration tubing system shown with a single fluid source and multiple (two) delivery devices;

FIG. 2 is a side elevation view of the one-to-many branching component of the infiltration tubing system shown in FIG. 1;

FIG. 3 is a side view of an alternative embodiment of the one-to-many branching component shown in FIG. 2;

FIG. 4 is a side elevation view of an alternative embodiment of a one-to-many infiltration tubing system shown with a single fluid source and multiple (two) delivery devices; and

FIG. 5 is a side elevation view of the one-to-many branching component of the infiltration tubing system shown in FIG. 4.

### DETAILED DESCRIPTION OF THE INVENTION

A one-to-many branching configuration for infiltration tubing is described herein. A one-to-many infiltration tubing branching configuration such as the one described herein allows for a dramatic decrease in the time required to complete an infiltration. This dramatic decrease in the infiltration time significantly decreases the degree of discomfort experienced by the patient during the infiltration process.

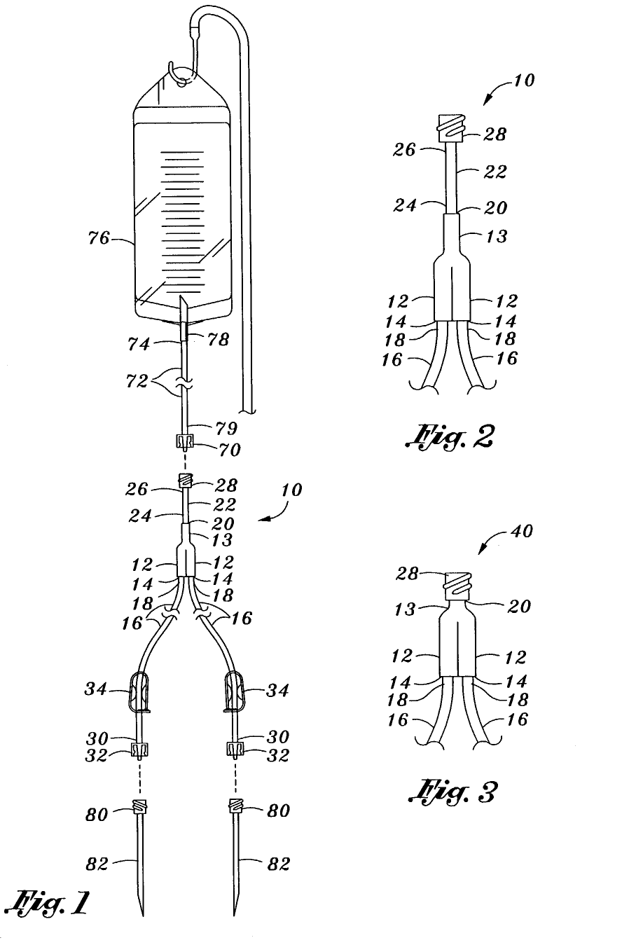

Referring now to the drawings wherein the showings are for purposes of illustrating preferred embodiments of the present invention only, and not for purposes of limiting the same, FIGS. 1-3 illustrate exemplary embodiments of a one-to-many branching configuration 10, 40 for infiltration tubing. The embodiments shown in FIGS. 1-3 use a parallel Y connector configured to be directly connected (e.g., glued) to infiltration tubing. FIGS. 4-5 illustrate an alternative (non-parallel) embodiment in which the one-to-many branching configuration 50 includes connectors for connecting to the infiltration tubing, e.g., using slip luers or luer locks.

Traditionally, (i.e., in a one-to-one configuration), IV lines 72 are attached at the proximal end 74 to the IV bag/bottle 76 by means of a spike (not shown) inserted through a rubber stopper 78 on the bottle/bag. The distal end 79 of the IV line 72 consists of a male luer connector 70 (Slip-Luer or Luer Lock). This male luer connector 70 is attached to a female luer connector of an IV catheter, hypodermic needle, or a length of IV extension tubing.

In exemplary embodiments, the one-to-many arrangement of IV tubing as shown in FIGS. 1-3 has one proximal inlet 13 and multiple (two or more) distal outlets 12.

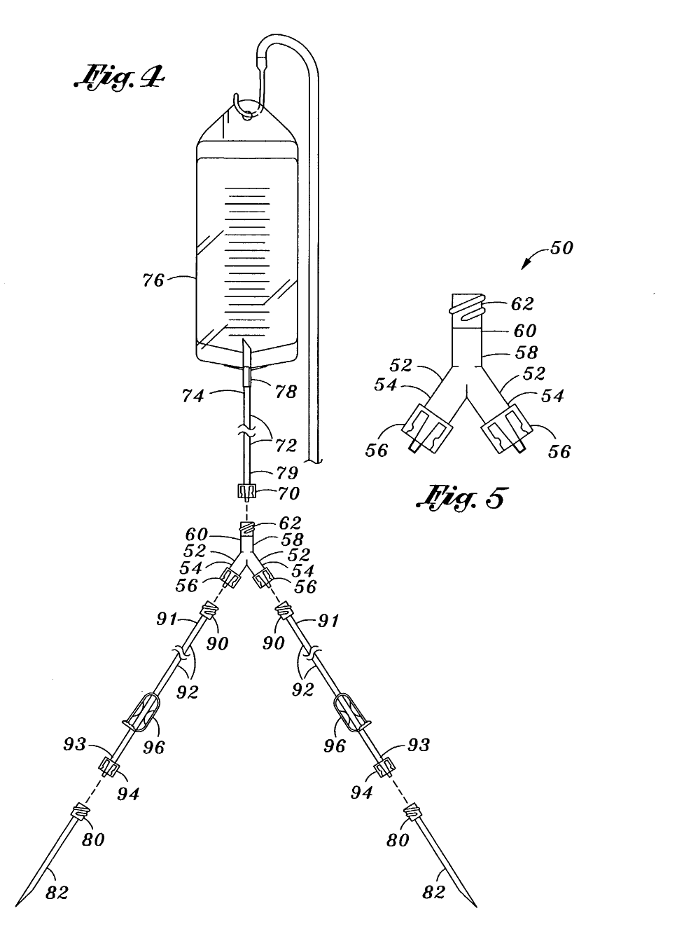

The distal end 20 of the proximal inlet 13 connects to a female connector 28, e.g., a luer connector. The proximal end of the one-to-many connector 10, 40 has either a female luer connector 28 (as shown) or is permanently attached (not shown) either to an IV extension tubing (e.g., twenty-four inches in length) or directly to IV tubing 72 attached to an IV bag 76. The distal end 20 of the proximal inlet 13 may be connected to the female connector 28 directly as shown in the one-to-many connector 40 shown in FIG. 3 or via a short length of IV tubing 22 as shown in the one-to-many connector 10 shown in FIGS. I and 2. In the embodiment shown in FIGS. 1 and 2, the distal end 20 of the proximal inlet 13 is connected to the proximal end 24 of the short tubing 22. In exemplary embodiment, the proximal end 24 of the short length of tubing 22 is glued into the distal end 20 of the proximal inlet 13 and the distal end 26 of the short length of tubing 22 is glued to female connector 28. The female connector 28 is configured to be connected to male connector 70 which is attached to the fluid source 76 as shown in FIG. 1 and described above.

Each of the distal outlets 12 leads to a delivery device 82, such as an infiltration cannula, a needle, etc. In exemplary embodiments, shown in FIGS. 1-3, the proximal ends 18 of multiple distal tubes 16 are glued directly to distal openings 14 of a one-to-many connector 10, 40. The distal ends 30 of each of these tubes 16 has a male connector 32. The male connector 32 is configured to connect to a female connector 80 attached to a fluid delivery device 82.

Each of the distal tubes 16 extending from the one-to-many connector 10, 40 is equipped with a simple on-off switch 34, such as a clamp or stopcock. This on-off switch 34 allows the clinician to temporarily stop all flow through the distal tube 16, remove the infiltration cannula 82 from the subcutaneous space and reinsert the cannula 82 into a new position while the infiltration process continues through the other cannulas 82 that remain in their subcutaneous positions.

FIGS. 4-5 illustrate an alterative embodiment of a one-to-many infiltration tubing configuration 50. The embodiment shown in FIGS. 4 and 5 has a traditional, i.e., non-parallel, “Y” shape as contrasted with the parallel “Y” shape of the embodiments shown in FIGS. 1-3.

In the embodiment shown in FIGS. 4-5, the connector 50 has one proximal inlet 58 and multiple (two or more) distal outlets 52. The distal end 60 of the proximal inlet 58 includes a female connector 62, e.g., a luer connector. As described above, this female connector 62 can be attached to an IV extension tubing or directly to IV tubing 72 attached to an IV bag 76.

The connector 50 shown in FIGS. 4 and 5 includes multiple (two or more) distal outlets 52. Each of the distal outlets 52 leads to a delivery device 82, such as an infiltration cannula, a needle, etc. In exemplary embodiments, shown in FIGS. 4-5, male connectors 56 are attached to the proximal ends 54 of each of the distal outlets 52. These male connectors 56 are configured to be connected to female connectors 90 that are connected to distal tubing 92. The distal tubing 92 has female connectors 90 at the distal end 91 of the distal tubing 92 and male connectors 94 at the proximal end 93 of the distal tubing 92. The male connectors 94 can be connected to a delivery device 82, such as an infiltration cannula or needle 82 having a female connector 80.

Each of the distal tubes 92 connected to the one-to-many connector 50 is equipped with a simple on-off switch 96, such as a clamp or stopcock, such as the one described above with reference to the embodiments shown in FIGS. 1-3.

Additional modifications and improvements of the present invention may also be apparent to those of ordinary skill in the art. Thus, the particular combination of parts described and illustrated herein is intended to represent only a certain embodiment of the present invention, and is not intended to serve as a limitation of alternative devices within the spirit and scope of the invention.

1. An infiltration tubing device, comprising:
a fluid source;
a one-to-many branching tube, comprising:
an inlet configured to receive a fluid supplied from the fluid source; and
at least two outlets, from which the fluid received from the fluid source flows out of the one-to-many branching tube; and
at least two infiltration cannulae connected to the respective outlets of the one-to-many configuration, wherein the infiltration cannulae are operative to infiltrate the fluid to a patient simultaneously.

2. The infiltration tubing device as recited in claim 1, wherein the inlet is configured to connect to the fluid source with an IV tube disposed therebetween.

3. The infiltration tubing device as recited in claim 1, wherein the at least two outlets are substantially parallel in relation to each other.

4. The infiltration tubing device as recited in claim 1, wherein the inlet is attached to an inlet female connector and the fluid source comprises a fluid source male connector, the proximal inlet female connector configured to connect to the fluid source male connector.

5. The infiltration tubing device as recited in claim 4, wherein the inlet female connector is attached to the inlet via a short tubing.

6. The infiltration tubing device as recited in claim 5, wherein the short tubing is glued to the inlet female connector and the inlet.

7. The infiltration tubing device as recited in claim 4, wherein the inlet female connector and the fluid source male connector are luer connectors.

8. The infiltration tubing device as recited in claim 7, wherein the inlet female connector and the fluid source male connector have a luer connection.

9. The infiltration tubing device as recited in claim 7, wherein the inlet female connector and the fluid source male connector have a luer lock connection.

10. The infiltration tubing device as recited in claim 1, wherein each of the at least two outlets is configured to connect the respective cannulae with a tube disposed therebetween.

11. The infiltration tubing device as recited in claim 10, wherein each of the at least two outlets is glued to the respective tube.

12. The infiltration tubing device as recited in claim 10, wherein each of the at least two outlets comprises an outlet male connector.

13. The infiltration tubing device as recited in claim 12, wherein each of the respective tubes comprises a tube female connector, configured to connect to the respective outlet male connector of the outlets.

14. The infiltration tubing device as recited in claim 13, wherein the outlet male connectors and the tube female connectors are luer connectors.

15. The infiltration tubing as recited in claim 14, wherein the outlet male connectors and the tube female connectors have a luer slip connection.

16. The infiltration tubing as recited in claim 14, wherein the outlet male connectors and the tube female connectors have a luer lock connection.

17. The infiltration tubing as recited in claim 10, wherein the tubing comprises an on-off switch configured to temporarily stop flow through the distal tubing.

18. The infiltration tubing as recited in claim 17, wherein the on-off switch is a clamp.

19. The infiltration tubing as recited in claim 17, wherein the on-off switch is a stopcock.

20. A one-to-many infiltration connector used for delivering a fluid from a single fluid source to multiple sites of a patient, the connector comprising:
one inlet in fluid communication of the single fluid source; and
a plurality of outlets operative to deliver the fluid to the multiple sites of the patient simultaneously.

21. The one-to-many infiltration connector as recited in claim 20, further comprising at least a valve at each of the outlets.

22. A one-to-many infiltration connector used for delivering a fluid from a single fluid source to multiple sites of a patient, the connector comprising:
one inlet for receiving the fluid from the single fluid source; and
a plurality of outlets in communication with the multiple sites of the patient, wherein when communication between any of the outlets and the site of the patient is interrupted, the remaining outlets are operative to continue delivery of the fluid to the corresponding sites of the patient.

23. The one-to-many infiltration connector as recited in claim 22, further comprising a plurality of cannulae providing fluid communication between the outlets and the multiple sites of the patient.
